(19) United States
(12) Patent Application Publication
(10) Pub. No.: US 2008/0286746 A1
(43) Pub. Date: Nov. 20, 2008

(54) PERFUSION APPARATUS AND METHOD

(75) Inventors: Ramon E. Poo, Miami, FL (US); Camillo Ricordi, Miami, FL (US)

Correspondence Address: AKERMAN SENTERFITT, P.O. BOX 3188, WEST PALM BEACH, FL, 33402-3188, US

(73) Assignees: Biorep Technologies, Inc., Miami, FL (US); University of Miami, Miami, FL (US)

(21) Appl. No.: 11/750,147

(22) Filed: May 17, 2007

(51) Int. Cl. A01N 1/00; C12M 1/00

(52) U.S. Cl. 435/1.1; 435/284.1

(57) ABSTRACT

A perfusion apparatus for perfusing an organ includes a pump for pumping perfusion liquid though a perfusion liquid conduit, the conduit having an outlet. A pressure sensor senses the pressure of the perfusion liquid downstream of the pump and upstream of the outlet. A controller controls the output of the pump responsive to the pressure, so as to keep the pressure of the liquid reaching the organ substantially constant.

### CROSS-REFERENCE TO RELATED APPLICATIONS

Not applicable.

### STATEMENT REGARDING FEDERALLY SPONSORED RESEARCH OR DEVELOPMENT

Not applicable.

### BACKGROUND OF THE INVENTION

There are currently promising applications for isolated cells from parent organs, such as the liver, spleen, kidney, adrenal, and pancreas. For example, therapies have been developed for the clinical application of isolated pancreatic cells called the Islets of Langerhans as a treatment for diabetic patients. Patients with diabetes either have Islets of Langerhans that do not function properly, and therefore, do not produce enough insulin, known as Type 2 Diabetes, or do not have Islet cells at all, Type 1 Diabetes. Therapies have been developed by which functioning Islets of Langerhans cells are transplanted into diabetic patients to restore the insulin producing ability of the pancreas. Such therapies require isolated Islet of Langerhans cells, but these cells must be isolated while still viable. Viable isolated cells are mostly obtained from organs of the very recently deceased. The apparatus and method for isolating the cells must be able to extract isolated cells with as little damage to the cells as possible.

Many different methods and approaches have been attempted to isolate individual cells from their respective parent organs. Prior methods have produced isolated cells with some cell destruction. This cell destruction can result from the relatively severe mechanical stimulation that is used to isolate cells from an organ. For example, enzymatic digestion is used to separate cells of interest from an organ, such as Islet cells from a pancreas. The process can include the mechanical disruption of the organ, as by cutting or chopping, in order to harvest the cells of interest. The result can be damage and mortality to some portion of the cells of interest.

One method that attempts to overcome the loss of damaged cells due to relatively severe mechanical stress is described in U.S. Pat. No. 5,079,160 to Lacy, et al. The method disclosed by Lacy, et al. comprises the steps of: placing an organ or a piece of an organ in a digestion chamber along with marble agitators; distending the organ or a piece of the organ with physiologically compatible medium containing a protease; continuously recirculating that medium; and separating the isolated cells. Agitators, such as those described in U.S. Pat. No. 6,833,270, greatly increase the amount of undamaged cells obtained through isolation without reducing the quality of the isolated cells obtained by gently agitating the organ. Moreover, the marbles are an appropriate size, weight, and density for obtaining beneficial results as compared to other agitators of varying size, weight, and density which can cause severe mechanical disruption of the organ tissue resulting in some cells being destroyed.

The protease utilized by the Lacy, et al. method and other similar methods requires optimal temperatures for appropriate activity of the protease. This temperature depends on the particular protease and other operating conditions, however, the temperature is usually elevated relative to room temperature. Therefore the medium must be heated to the desired temperature, for example, 32° C. for some collegenases. Various devices for heating liquids are known, including heat exchange with other fluids and contact with heating elements. It is desirable that such devices for use in cell extraction provide very precise temperature control, sterility, and ease of use.

It has been found that perfusion of organs is necessary prior to digestion of the organs by methods such as taught by Lacy et al. Perfusion is the flowing of a perfusion liquid through the organ. Perfusion distends the organ to open canals, pockets and spaces within the organ, to allow digestive enzymes to thoroughly contact the cells of interest within the organ during the enzymatic digestion step. Perfusion has been performed manually using a syringe. The perfusion liquid is placed into the syringe, and a catheter connected to the outlet of the syringe is placed into the organ. The operator then operates the syringe to inject perfusion liquid into the organ. The operator usually attempts to maintain a substantially constant fluid pressure during the perfusion process by applying a constant manual pressure to the plunger of the syringe. Due to the imprecise nature of the friction and fluid characteristics of a syringe, the inherent inaccuracies of human sensation, and the variable flow characteristics of the perfusion liquid through the organ, it is difficult to maintain a substantially constant fluid pressure during perfusion.

### SUMMARY OF THE INVENTION

A perfusion apparatus has a pump for pumping perfusion liquid though a perfusion liquid conduit. The conduit has an outlet. A pressure sensor is provided for sensing the pressure of the perfusion liquid downstream of the pump and upstream of the outlet. A controller provides for control of the output, such as flow rate, of the pump responsive to the fluid pressure, so as to keep the pressure substantially constant.

The pump can develop pressures up to 500 mmHg or more depending on the application. The controller has a set point pressure, and controls the pump to maintain the perfusion liquid pressure substantially at the set point pressure. In one embodiment, the controller and the pump maintain the pressure ±5 mmHg from the set point pressure. The set point pressure can be between 80 mmHg and 180 mmHg.

The controller can have a timer, and at least a second set point pressure. The controller causes the pump to maintain the perfusion liquid pressure at the first set point pressure for a time period, and following the time period causes the pump to maintain the perfusion liquid pressure at the second set point pressure.

A method for perfusing an organ with a perfusion liquid includes the steps of providing a perfusion liquid conduit, the liquid conduit being connected to a pump; providing a pressure sensor for sensing the pressure of the perfusion liquid downstream of the pump; providing a controller for controlling the operation of the pump responsive to the pressure; positioning an outlet from the conduit in the organ; operating the pump to flow perfusion liquid through the organ; sensing the pressure of the perfusion liquid downstream of the pump, and controlling the operation of the pump responsive to the pressure so as to maintain the pressure of the perfusion liquid downstream of the pump and upstream of the organ substantially constant.

An automatic fluid heating assembly includes a conductive conduit. A first clamp is provided for engaging the conduit at a first position, and a second clamp is provided for engaging the conduit at a second position. The clamps have respective first and second electrical contacts. A voltage source applies a voltage across the conduit from the first electrical contact and the first position to the second electrical contact and the second position, the voltage causing resistance heating of said conduit. The conductive conduit has an inlet opening and an outlet opening, whereby fluid flowing through the conductive conduit from the inlet opening to the outlet opening will absorb heat from the conduit between the first position and the second position.

### BRIEF DESCRIPTION OF THE DRAWINGS

There is shown in the drawings embodiments which are presently preferred, it being understood, however, that the invention can be embodied in other forms without departing from the spirit or essential attributes thereof.

FIG. 1 is a perspective view of a perfusion apparatus according to the invention.

FIG. 2 front elevation.

FIG. 3 is a rear elevation.

FIG. 4 is a left side elevation.

FIG. 5 is a right side elevation.

FIG. 6(A) is a perspective view of a perfusion tray according to the invention.

FIG. 6(B) is a cross section of the perfusion tray of FIG. 6(A).

FIG. 7 is a perspective view partially broken and partially in phantom, of a thermocouple according to the invention.

FIG. 8 is a front view of a perfusion system according to the invention.

FIG. 9 is a screen shot of a control display of pressure information.

FIG. 10 is a screen shot of a control display of temperature information.

FIG. 11 is a control display of time-programmed perfusion.

FIG. 12 is a perspective view of a heating coil assembly according to the invention.

FIG. 13 is a front elevation.

FIG. 14 is a screen shot of a dual perfusion control display for a computer.

### DETAILED DESCRIPTION OF THE INVENTION

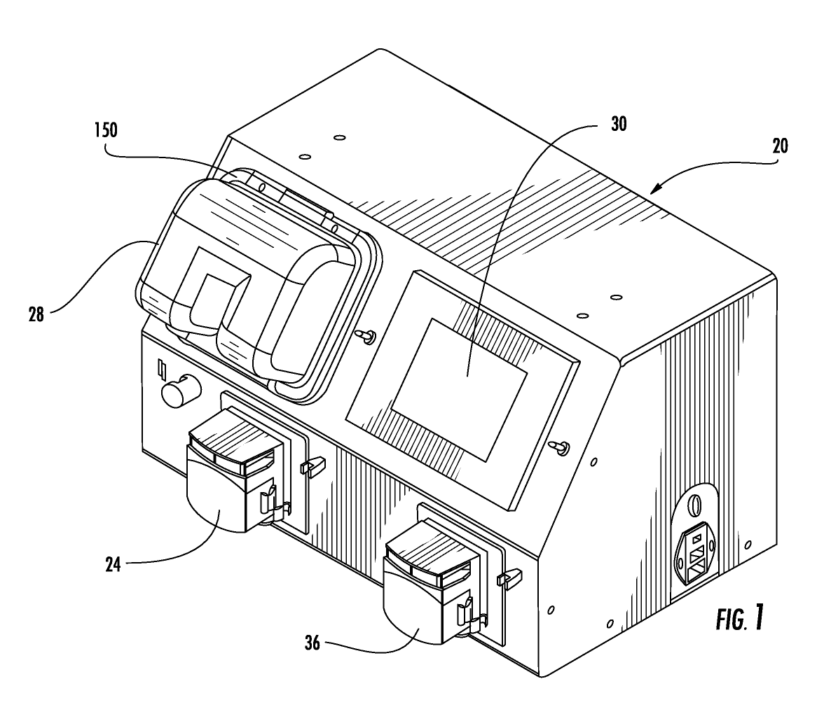

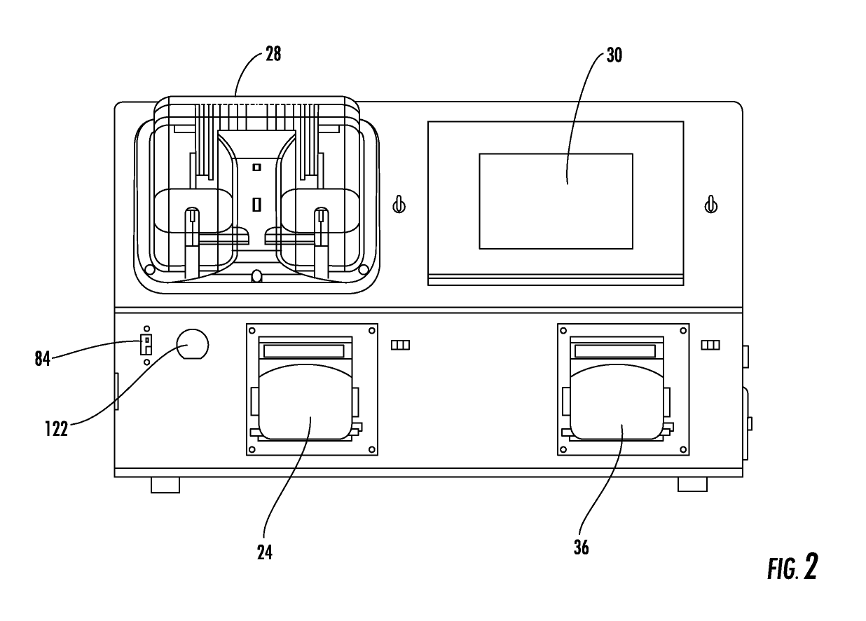

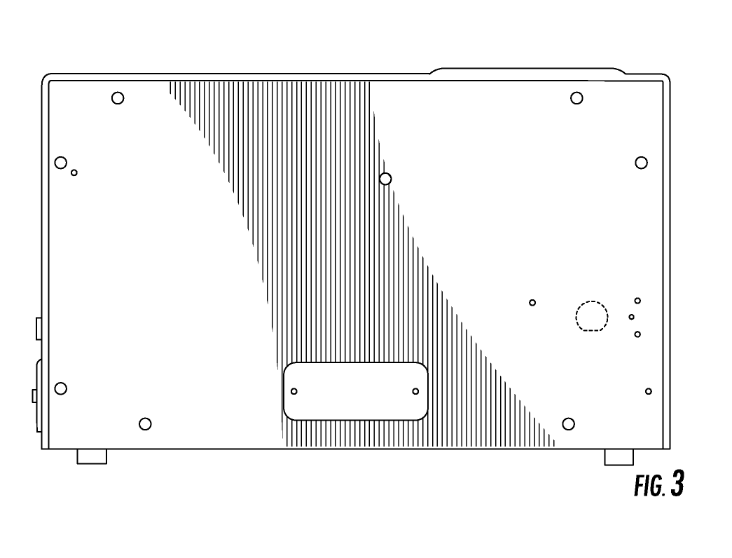

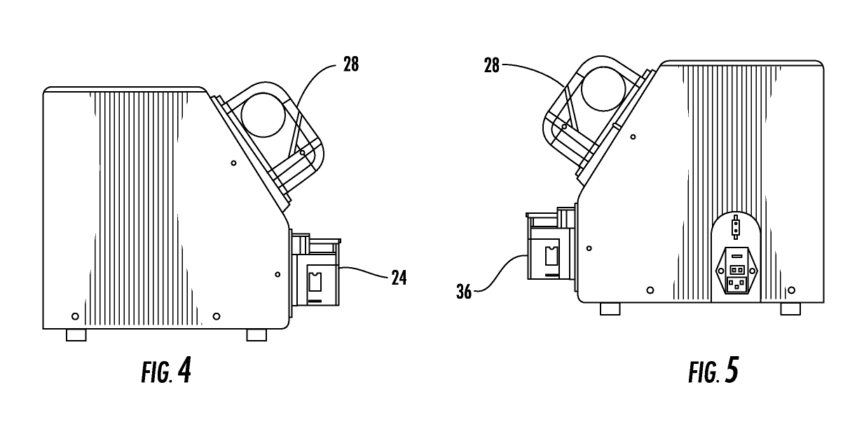

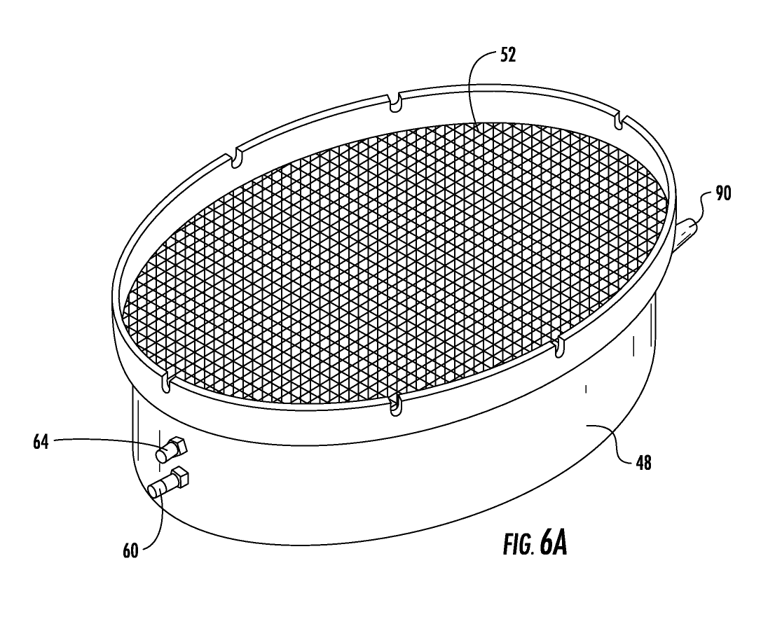

There is shown in FIGS. 1-5 a perfusion apparatus 20 according to the invention. The perfusion apparatus 20 includes at least one pump assembly 24 and at least one heating assembly 28. At least one pressure sensor is provided for sensing the pressure of liquid downstream of the pump assembly 24. The pressure sensor provides data to at least one controller, the controller providing a control signal to the pump assembly 24 so as to maintain the pressure at substantially a constant value, which can be a set point value. The controller, or a separate controller, can also be provided to control operation of the heating assembly 28 so as to provide a desired fluid temperature. The operator can interface with the controller through suitable structure such as a touch activated control screen 30. Computer control from a remote computer is also possible. A second pump assembly 36 can be provided to permit the perfusion of another organ, or another section of the same organ. While a single heating assembly 28 is shown, it is within the scope of the invention to have multiple heating assemblies.

The pump assemblies 24 and 36 are connected by suitable conduits, such as flexible tubing, to the organ. A catheter can be used to provide infusion of the liquid into the organ. The catheter can be inserted in any suitable location. In the case of the perfusion of a pancreas, the catheter is inserted in the pancreatic duct.

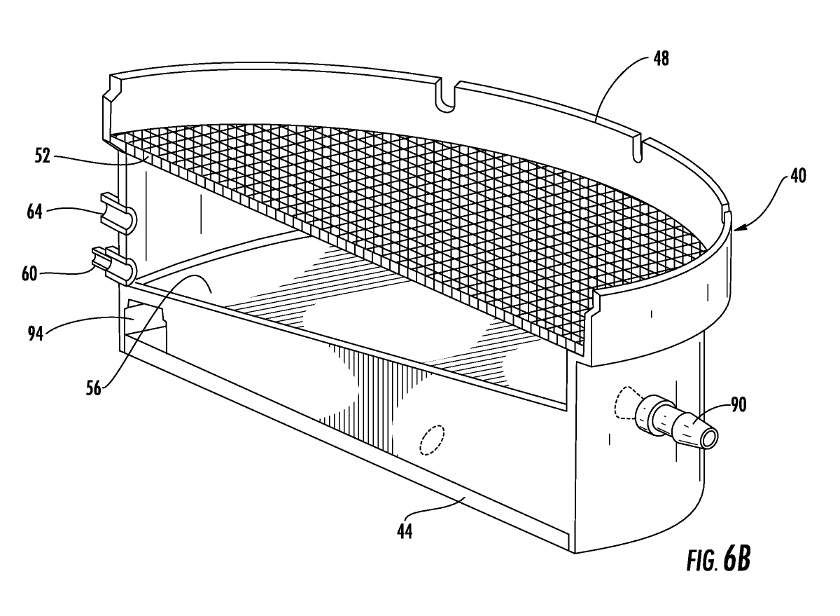

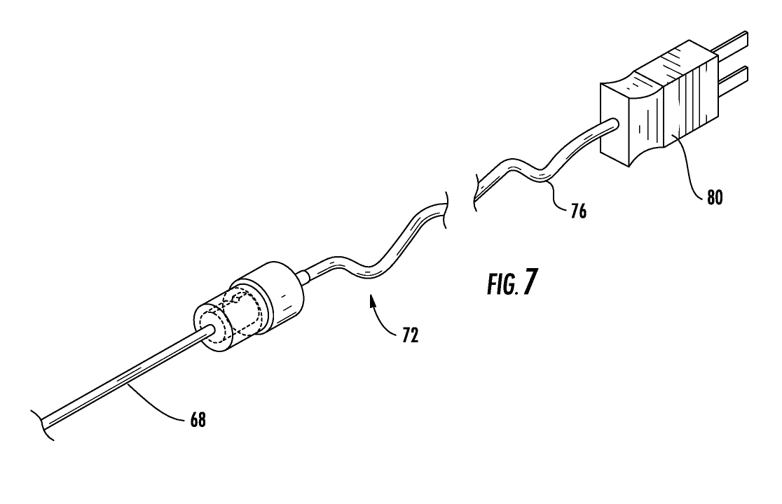

The organ can be placed on a suitable support which can also collect the perfusion liquid as it flows out of the organ, and preferably recycle this liquid to the pump assemblies 24 and 36. A suitable perfusion tray 40 is shown in FIGS. 6(A)-(B). The perfusion tray 40 has a base 44 and side walls 48. A porous support surface 52 can be provided and rests over the base 44 as on suitable supports provided by the side walls 48. The porous surface 52 permits perfusion liquid leaving the organ to pass through, where it is collected and directed by an incline surface 56 to an outlet 60. A thermocouple port 64 can receive a probe 68 of a suitable temperature sensor such as the thermocouple 72 (FIG. 7). The thermocouple input can be a type T thermocouple. The thermocouple 72 can be a disposable Luer lock thermocouple with a mini plug connector. A data lead 76 and plug 80 of the thermocouple 72 can be connected to a suitable input 84 on the perfusion apparatus 20, such that temperature information is provided to the controller. The perfusion tray 40 can also have ports 90 connecting to one or more cooling channels 94. Flow of a suitable cooling liquid such as water through the cooling channel 94 will conductively cool the perfusion liquid as it flows over surfaces of the perfusion tray 40. This will keep the perfusion liquid cold, and any digestive enzyme in the perfusion liquid inactive.

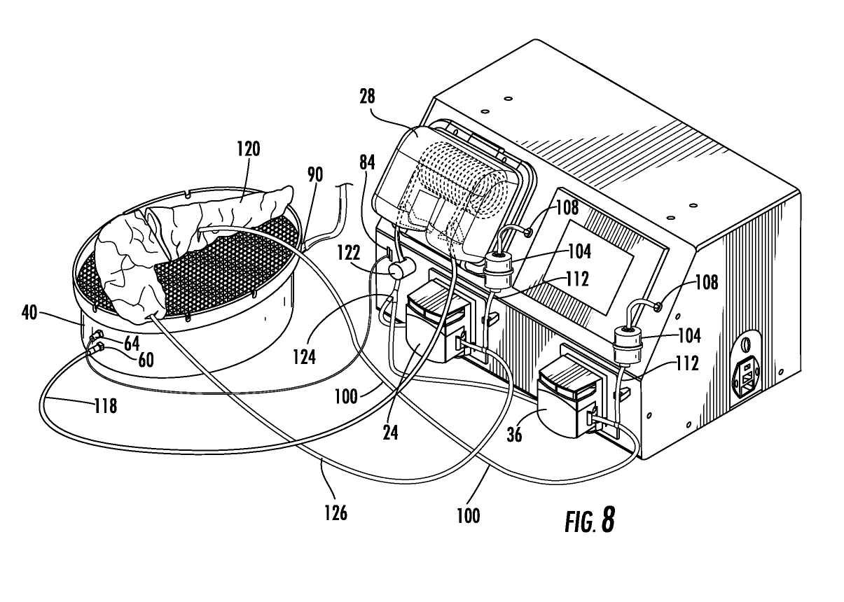

A typical system configuration is shown in FIG. 8. The perfusion apparatus 20 has connected thereto a perfusion liquid supply conduit 100, which receives liquid from the heating assembly 28. The supply conduit 100 can be routed through an infrared sensor 122 to sense the temperature of liquid leaving the heating assembly 28. The supply conduit 100 is connected to the pump 24 and/or pump 36. The pumps 24 and 36 can be a peristaltic pump, such that connection of the supply conduit 100 can be accomplished by threading the supply conduit 100 into the pump. Other pump designs are possible and the connections should be suited to the pump that is used. A branch 112 of conduit connects the supply conduit 100 to a pressure transducer protector 104 and a pressure input port 108, and thereby to a pressure transducer that is located within the machine. The pressure transducer protector 104 provides an air reservoir to stabilize the pressure readings. The supply conduit 100 then is connected to the organ 120 such as a pancreas, by suitable structure such as a catheter. The organ can rest on a suitable support such as the perfusion tray 40. A return conduit 118 is connected between the outlet 60 of the perfusion 40 and the heater assembly 28. In the case where a second pump 36 is also used, the supply conduit 100 can branch at a Y-connector 124 to a second liquid supply conduit 126, which can connect to another organ or another portion of the same organ as shown. The second liquid supply conduit 126 can also connect to a branch 112 of conduit which connects the second liquid supply conduit 126 to another pressure transducer protector 104 and another pressure input port 108, and thereby also to a pressure transducer that is located within the machine.

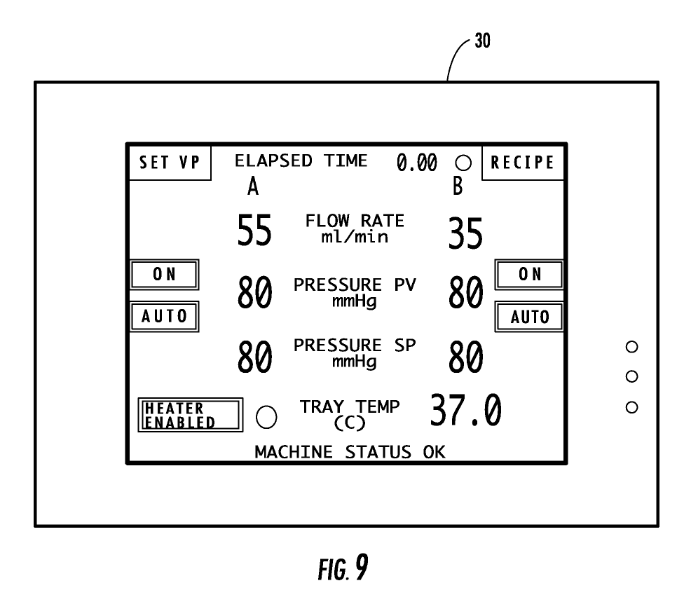

The perfusion apparatus can be operated in either a manual or automatic mode. As shown in FIG. 9, the touch screen 30 can be operated from either an AUTO or MANUAL mode by pressing the appropriate screen icon. In the MANUAL mode, the pressure is adjusted manually by controlling the flow rate through the pump responsive to pressure readings. In the AUTO mode, a pressure set point “PRESSURE SP” is used to set the desired pressure. The present value pressure “PRESSURE PV” is also indicated. The pump speed is increased by a controller when the PRESSURE PV reading is less than the PRESSURE SP, and the pump speed is decreased when the PRESSURE PV reading is greater than the PRESSURE SP reading.

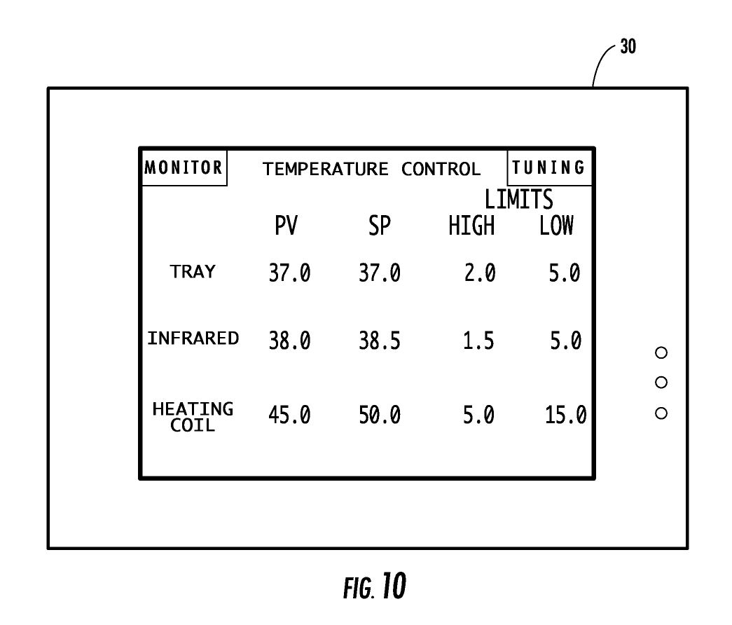

Temperature of the perfusion liquid can be controlled using the touch screen 30 display shown in FIG. 10. Alternatively, a different physical screen can be used to display the temperature set points at the perfusion tray 40, the heating assembly 28 and the conduit 100. Temperature at the perfusion tray 40 as measured by the thermocouple 72 is used to control the heating process. Temperatures at the heating assembly 28, measured at the heating coil, and conduit 100, measured by an infrared sensor 122, are monitored for purposes of safety, for example, where the heater is on but no liquid is flowing these sensors will cause the controller to turn the heating assembly 28 off. An upper limit is determined and input as a set point SP, and should the present value PV exceed this set point, the heating assembly 28 is automatically turned off. Temperatures can also be monitored elsewhere in the system.

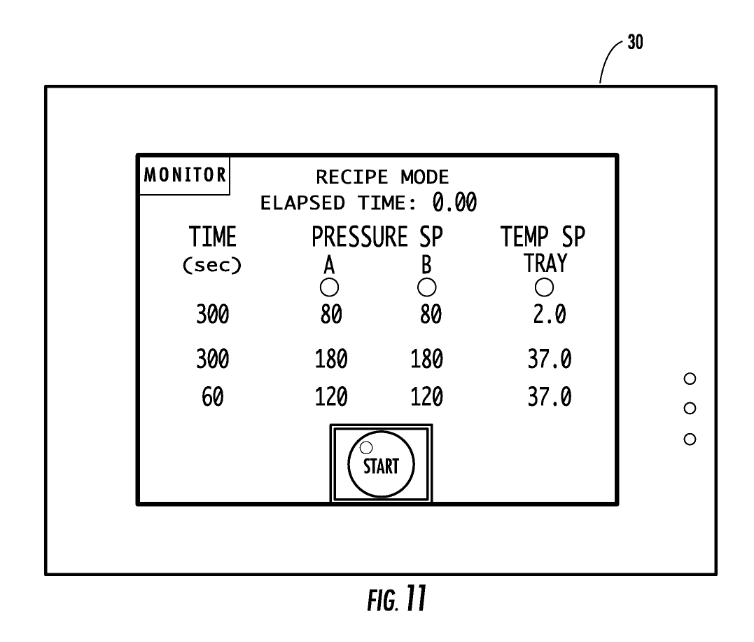

The touch screen display 30 shown in FIG. 11 is used in recipe mode to create time-varying protocols. Pressure and temperature set points can be entered as a function of time such that, for example, a cold perfusion liquid can be flowed through the organ at a temperature that will not activate a digestive enzyme in the liquid (2.0° C. for the first 300 seconds in FIG. 11), and then the temperature can be raised to subject the organ to the activated enzyme for a given time (37.0° C. for the next 300 seconds in FIG. 11, which can be followed by another 60 seconds at 37.0° C. while pressure settings are changed). The recipe mode helps to ensure reproducibility with preset pressure and temperature settings. Different protocols can be created. For example, there could be a protocol for fatty pancreases, or for pancreases or other organs that weigh more than a certain weight, or less than a certain weight. Desired protocols for organs with certain characteristics could be programmed and used again for organs with the same or similar characteristics. As new protocols are discovered, or existing protocols are refined and improved, they can stored in the system and recalled for later use.

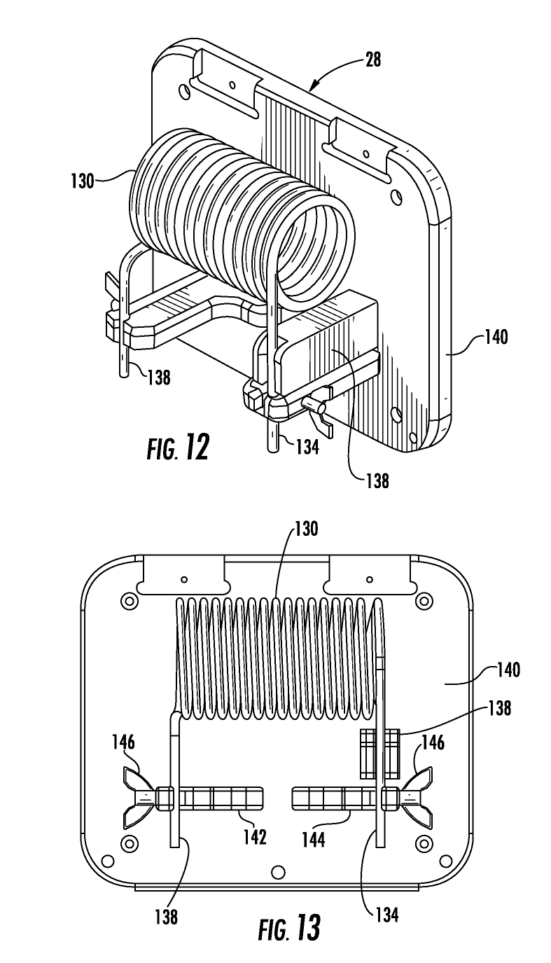

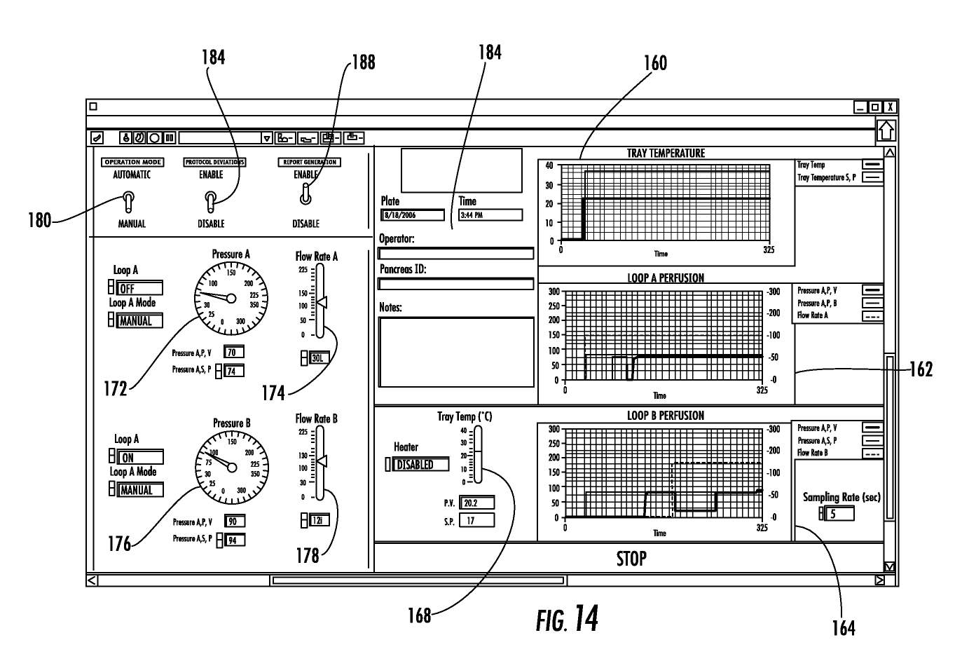

The heating assembly can be of any suitable construction. A heating assembly 28 according to one aspect of the invention is shown in FIGS. 12-13. A heating coil 130 is constructed from a conduit having an inlet 134 and an outlet 138 and permits the passage of fluid through the interior of the conduit. The heating coil 130 is constructed from a conductive material. Electrical contacts are provided to cause an electrical current to flow through the conduit, which will cause the temperature of the coil to rise. Heat will be conducted from the coil to the perfusion liquid flowing within, causing the temperature of the perfusion liquid to rise. By controlling the applied voltage to the coil 130, as by turning the applied voltage on and off, the heating of the coil and the perfusion liquid can be carefully controlled. Other means for controlling the heating of the coil, such as by varying current or applied voltage, can alternatively be used.

The coil 130 can have any number of turns, or can be without turns. It is only necessary that the coil 130 have sufficient length to impart adequate heating to the perfusion liquid. The conduit 130 can be straight, curved or in any other suitable shape. A coil shape permits a length of coil to be provided in a confined space without angles or corners in the conduit such as might impede fluid flow. The coil characteristics can vary with such characteristics as the nature of the perfusion liquid, inlet liquid temperature and desired outlet temperature, the heat capacity of the liquid, the resistance of the coil material, flow rate of the liquid, the applied voltage, and the current flowing through the coil. The electrical contacts can be in any suitable form which both engage and hold the coil in place, and provide an electrical contact to a suitable voltage source. Suitable fastening structure can be used to adjust the clamping strength and to provide a good electrical contact. A heating coil thermocouple can also be located in at least one of the clamps to measure temperature at this location.

The conduit 130 can be mounted on any suitable support, such as base 140. The base 140 can be made from an insulating material so as not to conduct current away from the conduit 130. Structure for engaging the conduit 130 is provided. The engagement structure can be engagement arms 142, 144 which detachably engage the heating conduit 130. This will permit removal of the conduit 130 for replacement or cleaning. The engagement arms 142, 144 can have electrical contacts or can be made from an electrically conductive material such as metal. Fastening structure such as wing head thumbscrews 146 can be provided and securely tightened so there is good contact between the coil and the engagement arms 142, 144. The surface of the metal coil makes contact with a spring-loaded thermocouple 138. This thermocouple is used to monitor the surface temperature of the coil, which can be used to stop the heating process if the temperature exceeds a set point, such as a do-not-exceed safety set point. A cover 150 is provided. Suitable circuitry can be provided such that the conduit 130 will be OFF if the cover 150 is open and the conduit 130 is exposed. This will prevent accidental burns.

The conducting material can be any suitable conducting material such as a metal. One such metal is stainless steel. Other conductive materials such as aluminum can be used, or non-conductive materials that are lined with a conductive material or have conductive materials secured thereto or embedded therein.

A voltage source is connected to the contacts such that a potential difference will be applied to the contacts and across a length of the heating conduit 130. The voltage that is applied across the conduit 130 can be varied. The applied voltage is generally low, between about 5 VAC and about 24 VAC. The applied voltage will depend operational parameters such as flow rate, inlet temperature, desired outlet temperature, the heat capacity of the fluid, the material resistance and the dimensions of the coil including inside diameter, outside diameter, and length.

In one embodiment, the elements of the automatic heater are a stainless steel coil (0.150″ ID×0.180″ OD×10 ft), a buck and boost transformer (0.250 VA) (115 VAC in−12 VAC out), a relay (solid state for more accurate control period), and a temperature controller or PLC (programmable logic controller temperature control card). However, the conduit 130 could also be made of aluminum with other dimensions and thus with a different size transformer.

A low voltage such as 12 VAC that is applied across the two ends of the coil can cause a high current of approximately 25 A to flow through the metal coil, and the temperature of the coil rises. By turning on/off the primary voltage of the transformer, the temperature of the fluid flowing through the coil can be accurately controlled. Since the coil has very little mass, its temperature rises and falls quickly response to heat transfer. A standard temperature controller is capable of turning the relay on/off.

The heating assembly 28 can keep the temperature of the perfusion liquid constant at any temperature above room temperature. The heating assembly 28 can also be used to control the temperature of other fluids, such as fluid flowing through an organ digestion chamber. Any standard temperature controller can control the conduit 130 to maintain the fluid temperature at or near a desired set point, such as at 32.0° C., the optimal temperature for the enzyme to digest the pancreas during an islet isolation procedure.

Silicone tubing or other conduit can be connected to the outlet of the heating coil 130. From the outlet, the tubing can be routed through an infrared thermocouple bracket. This non invasive sensor will shut off the heater in case the temperature of the fluid has reached a maximum allowable limit. The temperature from the thermocouple 72 is used to control the process and the infrared temperature controller acts as a high limit temperature switch. The thermocouple 72 provides feedback to a suitable temperature controller and depending on this temperature, the heating assembly will turn ON and OFF accordingly. Therefore, the infrared sensor set point is set at the maximum allowable temperature.

FIG. 14 is a screen shot on an interface for controlling the perfusion apparatus from a computer. This screen, suitable for a personal computer (PC), allows full control of the perfusion apparatus, and as shown, for dual perfusion control. Displays are provided for tray temperature 160, loop A perfusion 162 and loop B perfusion 164. Displays can also be provided for pressure A 172, flow rate A 174, pressure B 176, and flow rate B 178. Displays for the selection between auto-manual 180, enable-disable protocol deviation 184, and enable-disable report generation 188 can also be provided. Other displays are possible. The computer can be remotely located relative to the perfusion apparatus. Also, the recipe parameters can be inputted into the apparatus through standard spreadsheet programs suitable for PCs. Data acquisition similarly can be accomplished through PC based programming, and plotted, stored or otherwise processed by the PC. Changes in the protocols can be recorded and the results analyzed by PC based programs.

In practice, the perfusion device 20 is used to automatically pump a fluid at a constant pressure. Typical pumping pressures are in the range of 0-300 mmHg. Flow rates can range from 0-300 ml/min. As the pressure in the organ starts to build up, the machine automatically adjusts the flow rate to maintain a constant pressure SP. The apparatus can be used in conjunction with a disposable perfusion tray 40. The perfusion tray 40 and tubing set can be separately packed and sterilized. The perfusion tray 40 can be disposable and should then only be used once.

The apparatus itself can contain two or more pumps, pressure sensors, touch screen, and a programmable logic controller (PLC) so as to simultaneously perfuse different organs or different portions of the same organ. Each pump can be run independently or together in manual or automatic mode. The heater assembly 28 can be manually controlled by pressing an on/off button on the screen. In manual mode, the flow rate is adjusted by turning the dial until the desired flow rate and/or pressure is achieved. In automatic mode, the desired pressure set point on the pressure monitor can be selected by pressing up or down on appropriate keys. The flow rate will automatically adjust itself to maintain a constant pressure. The user can increase or decrease the pressure set point at any time as desired. Flow rate can be controlled by an input on the touchscreen or by other means such as a pop-up keypad.

The pump assemblies 24 and 36 are started after it is determined that the system is full of fluid. The heating conduit 130 should make good contact with the engagement arms 142, 144 and the heater cover 150 should be fully closed. The tubing extending from the outlet of the heating coil should go through the infrared sensor 122. The thermocouple 72 is connected to the front panel. If the thermocouple 72 is not connected, the heating assembly will not operate.

The thermocouple 72 is connected to the thermocouple fitting 64 of the perfusion tray 40. The tubing is connected to the cooling ports 90 and cooling water is recirculated at low pressure.

Starting from a cool temperature, the heater toggle switch is moved to the ON position. The temperature will now start rising in a controlled fashion and will reach the temperature set by the user on the controllers. Once this temperature is reached, the heater controller will cycle ON and OFF to maintain the desired temperature. There should be no cold air blowing on the chamber, heating coil, or tubing as this can adversely affect the heating time necessary to reach the set point. If for any reason the pump needs to be stopped, the heater should be turned off as well. If this step is not taken, the heating coil temperature will rise and reach 50.0° C. (or whatever set point is set on that thermocouple). This step can be programmed into a PLC to prevent the coil from overheating (if pump is off, then the heater is off). This is why it is important that the heating coil is in contact with the thermocouple.

Similarly, the pressure set point can be adjusted using the screen of FIG. 9. The pressure sensor will provide to the controller a pressure reading PV which will be compared by the controller to the set point SP. The pumps operation will be adjusted, and the pressure will be adjusted accordingly to maintain pressure at or near, for example ±5 mmHg, from the set point pressure.

This invention can be embodied in other forms without departing from the spirit or essential attributes thereof and, accordingly, reference should be had to the following claims rather than the foregoing specification as indicating the scope of the invention.

We claim:

1. A perfusion apparatus, comprising:
a pump for pumping perfusion liquid though a perfusion liquid conduit, said conduit having an outlet;
a pressure sensor for sensing the pressure of the perfusion liquid downstream of said pump and upstream of said outlet; and,
a controller for controlling the flow rate of said pump responsive to said pressure, so as to keep said pressure substantially constant.

2. The perfusion apparatus of claim 1, wherein said pump develops pressure up to 500 mmHg.

3. The perfusion apparatus of claim 1, wherein said controller has a set point pressure, and controls said pump to maintain said perfusion liquid pressure substantially at said set point pressure.

4. The perfusion apparatus of claim 3, wherein said controller and said pump maintain said pressure ±5 mmHg from said set point pressure.

5. The perfusion apparatus of claim 3, wherein said set point pressure is between 80 mmHg and 180 mmHg.

6. The perfusion apparatus of claim 3, wherein said controller comprises a timer, and at least a second set point pressure, said controller causing said pump to maintain said perfusion liquid pressure at said first set point pressure for a time period, and following said time period causing said pump to maintain said perfusion liquid pressure at second set point pressure

7. A method for perfusing a biological material with a perfusion liquid, comprising the steps of:
providing a perfusion liquid conduit, said liquid conduit being connected to a pump;
providing a pressure sensor for sensing the pressure of said perfusion liquid downstream of said pump;
providing a controller for controlling the flow rate of said pump responsive to said pressure;
positioning an outlet of said conduit in said biological material;
operating said pump to flow perfusion liquid through said biological material;
sensing the pressure of said perfusion liquid downstream of said pump, and controlling the flow rate of said pump responsive to said pressure so as to maintain said perfusion liquid downstream of said pump and upstream of said biological material substantially constant.

8. The method of claim 7, wherein said biological material is an organ.

9. An automatic fluid heating assembly, comprising:
a conductive conduit;
a first clamp for engaging said conduit at a first position, and a second clamp for engaging said conduit at a second position, said clamps having respective first and second electrical contacts;
a voltage source for applying a voltage across said conduit from said first electrical contact and said first position to said second electrical contact and said second position, said voltage causing resistance heating of said conduit;
said conductive conduit having an inlet opening and an outlet opening, whereby fluid flowing through said conductive conduit from said inlet opening to said outlet opening will absorb heat from said conduit between said first position and said second position.
